(19) United States
(12) Patent Application Publication
(10) Pub. No.: US 2012/0015906 A1
(43) Pub. Date: Jan. 19, 2012

(54) USES OF BORTEZOMIB IN PREDICTING SURVIVAL IN MULTIPLE MYELOMA PATIENTS

(76) Inventors: John D. Shaughnessy, JR., Roland, AR (US); Bart Barlogie, Little Rock, AR (US); Pingping Qu, Issaquah, WA (US)

(21) Appl. No.: 13/138,099

(22) PCT Filed: Jan. 4, 2010
(86) PCT No.: PCT/US2010/000002

§ 371 Date: Sep. 23, 2011

Related U.S. Application Data
(60) Provisional application No. 61/204,154, filed on Jan. 2, 2009.

(51) Int. Cl. A61K 31/69; A61K 31/454; A61P 35/00; C40B 30/04

(52) U.S. Cl. 514/64; 506/9; 514/323

(57) ABSTRACT

The present invention provides a method of predicting outcome of treatment for multiple myeloma based on determining certain cytogenetic anomalies and considering gene expression profiling risks. Also provided are statistical methods employed to define variables independently impacting outcomes. Further provided is method of treatment of myeloma patients.

### CROSS-REFERENCE TO RELATED APPLICATIONS

This international application claims benefit of priority under 35 U.S.C. §119(e) of provisional application U.S. Ser. No. 61/204,154, the entirety of which is hereby incorporated by reference.

### FEDERAL FUNDING LEGEND

This invention was supported in part by National Institutes of Health CA55819. Consequently, the federal government has certain rights in this invention.

### BACKGROUND OF THE INVENTION

1. Field of the Invention

The present invention generally relates to the field of cancer research. More specifically, the present invention relates to predicting the outcome of treatments in multiple myeloma patients and potential resistance to drugs. By utilizing gene expression profiling, myeloma patients may know ahead of time, whether they are likely to be resistant to certain chemotherapeutic agents and whether specific therapeutic regimens would be beneficial.

2. Description of the Related Art

The mechanism of action and resistance to chemotherapy is poorly understood and measures of efficacy typically rely on clinical outcome data. Recent advances suggest that prospective gene expression profiling (GEP) can be used to more accurately define not only short-term but lasting treatment benefits. More particularly, in the case of multiple myeloma (MM), the prognosis of patients has been markedly improved by the discovery of novel agents that exert anti-tumor effects also indirectly by co-targeting the bone marrow stroma, such as immuno-modulatory drugs, thalidomide, and lenalidomide, and the proteasome inhibitor, bortezomib.

Emerging evidence suggests that the prognosis of patients with multiple myeloma is best captured by gene expression profiling analysis of CD138-purified plasma cells (PC). A 70-gene model using baseline gene expression signatures defines high risk in approximately 15% of newly diagnosed disease. This high-risk model is driven in large part by copy number sensitive gene expression changes resulting from gains of chromosome 1q21 and loss of 1p13. Amplification of chromosome 1q21-23, representing the only recurrent high-level copy number amplification in myeloma, encompasses over 10 Mb of DNA and 100 genes. Candidate genes within this interval include IL6R, MCL1, BCL9, CKS1B, and PSMD4, with the latter two being components of the 70-gene risk model. PSMD4 encodes a protein that is a regulatory component of the multi-subunit proteasome complex. Bortezomib, a first in class proteasome inhibitor, has been shown to improve outcomes in newly diagnosed multiple myeloma patients.

As mentioned above, baseline tumor cell gene signatures encompassing 70 and as few as 17 genes can discriminate risk groups of myeloma patients both in the untreated and previously treated settings. However, a subset of predicted low-risk cases followed an aggressive clinical course accompanied by a shift from 170-gene-defined low- to high-risk over time, either reflecting clonal evolution or outgrowth of aggressive clones present, but undetectable, at diagnosis. Accurately identifying this patient population is a first step in preempting transformation.

Changes in gene expression patterns of tumor cells following a short term in vivo challenge with a single chemotherapeutic agent might expose these latently aggressive cells. Unlike in vitro testing, clinical drug administration also allows for assessing tumor cell perturbation in the context of host interactions. Building on recent observations that clinical outcome in myeloma patients could be correlated with 48 hr gene expression profiling changes induced in vivo following a single administration of thalidomide, lenalidomide and dexamethasone, whether such short-term tumor-cell gene expression profiling and proteomic alterations could fine tune clinical outcome prediction beyond the well-established 17-gene-based baseline prediction model was examined.

It is now well-recognized that combination chemotherapy with bortezomib improves the outcome in patients treated with comparable therapies lacking this drug. Bortezomib's incorporation into Total Therapy 3 (TT3) has fundamentally changed the prognosis of patients with gene expression profiling-defined low-risk disease, so that the estimated 4-year survival rate is 85%. Of those patients achieving a complete response (CR), 90% have remained relapse-free. By contrast, the fate of the 15% of patients with high-risk myeloma remains dismal so that fewer than 50% survive 3 years.

High-risk multiple myeloma is defined by 70-gene expression model that is driven by increased expression of genes mapping to chromosomes 1q and reduced expression of genes mapping to 1p. Genome-wide copy number analysis by a-CGH revealed 1q21 as the sole amplification hotspot within the myeloma genome. There is an inverse correlation between 1q21 copy number and survival. Introduction of bortezomib in Total Therapy 3 lifted the prognosis threshold of 1q21 copy number from >=3 in Total Therapy 2 to >=4, suggesting that genes residing in the 1q21 amplicon may determine sensitivity of multiple myeloma cells to proteasome inhibition and other chemotherapeutics and the addition of bortezomib overcomes a level of this resistance not achieved with other drugs. Of over 100 genes in the 1q21 amplicon, the proteasome gene, PSMD4, is 1 of only 2 genes whose increased copy number-driven hyper-expression contributed to the high-risk designation of the 70-gene model.

Hyper-activation of proteasome genes such as PSMD4 provides a mechanistic explanation for the poor outcome of patients with the high-risk designation despite the addition of bortezomib in TT3. Indeed, polymorphisms and point mutations in PSMB5 have been associated with increased resistance to proteasome inhibition. Gene expression profiling of multiple myeloma tumor cells prior to and following in vivo thalidomide, dexamethasone and lenalidomide exposure is feasible and can identify genes whose change in expression is related to outcome. For example, upregulation of the glucocorticoid receptor following short-term in vivo exposure to dexamethasone was associated with improved long-term outcome in patients receiving Total therapy 2. Thus, molecular perturbation of glucocorticoid receptor, the target of dexamethasone, is a biomarker of this drug's efficacy in combination chemotherapy.

As bortezomib targets the proteasome and elevated expression of PSMD4 in baseline samples is related to poor outcome, short term in vivo exposure of tumor cells to Velcade could lead to a rapid genomic response to proteasome inhibition and that this readout would provide measures of sensitivity and resistance to bortezomib, and bortezomib containing therapies.

While the survival impact of new agents, such as bortezomib and its derivatives, is profound, there is still a dismal survival rate amongst a subpopulations of myeloma patients, including some patients initially diagnosed as low-risk. One of the first steps to treating these patients is to preemptively identify individuals who may be resistant to specific drug agents such as bortezomib.

The prior art is deficient in providing a method of identifying high-risk myeloma patients and predicting the chemotherapeutic resistance to specific drugs. The present invention fulfills this long-standing need and desire in the art.

### SUMMARY OF THE INVENTION

The present invention is directed to a method for predicting the likelihood of transformation from a low-risk prognosis to a high-risk prognosis for a subject with multiple myeloma, comprising obtaining a gene expression profile of tumor cell genes before administration of a chemotherapeutic agent, administering a single dose of a chemotherapeutic agent to the subject, obtaining a gene expression profile of tumor cell genes after administration of the chemotherapeutic agent and comparing the before and after gene expression profiles, where the upregulation of genes in the profile obtained after administration compared to the profile before administration is indicative of a likelihood of transformation to a high-risk prognosis.

The present invention is also directed to a method for identifying latently aggressive multiple myeloma tumor cells in a subject, comprising obtaining a first gene expression profile of multiple myeloma tumor cells in the subject, contacting the tumor cells with a single chemotherapeutic agent and obtaining a second gene expression profile of the multiple myeloma tumor cells after contact with the chemotherapeutic agent, where upregulation of genes in the second profile compared to the first profile is indicative that the tumor cells are latently aggressive tumor cells.

The present invention is also directed to a method for treating multiple myeloma in a subject, comprising determining if the multiple myeloma tumor cells in the subject are latently aggressive, designing a chemotherapeutic regimen comprising one or more anticancer agents effective to suppress activation of genes in the latently aggressive tumor cells and administering the one or more anticancer agents to the subject thereby treating the multiple myeloma.

The present invention is directed to a method for predicting the likelihood of transformation from a low-risk prognosis to a high-risk prognosis for a subject with multiple myeloma, comprising obtaining a gene expression profile of tumor cell genes before administration of a chemotherapeutic agent, administering a single dose of a chemotherapeutic agent to the subject, obtaining a gene expression profile of tumor cell genes after administration of the chemotherapeutic agent and comparing the before and after gene expression profiles, where the downregulation of genes in the profile obtained after administration compared to the profile before administration is indicative of a likelihood of transformation to a high-risk prognosis.

The present invention is also directed to a method for identifying latently aggressive multiple myeloma tumor cells in a subject, comprising obtaining a first gene expression profile of multiple myeloma tumor cells in the subject, contacting the tumor cells with a single chemotherapeutic agent and obtaining a second gene expression profile of the multiple myeloma tumor cells after contact with the chemotherapeutic agent, where downregulation of genes in the second profile compared to the first profile is indicative that the tumor cells are latently aggressive tumor cells.

The present invention is also directed to a method for treating multiple myeloma in a subject, comprising determining if the multiple myeloma tumor cells in the subject are latently aggressive, designing a chemotherapeutic regimen comprising one or more anticancer agents effective to activate genes in the latently aggressive tumor cells and administering the one or more anticancer agents to the subject thereby treating the multiple myeloma.

### BRIEF DESCRIPTION OF THE DRAWINGS

The appended drawings have been included herein so that the above-recited features, advantages and objects of the invention will become clear and can be understood in detail. These drawings form a part of the specification. It is to be noted, however, that the appended drawings illustrate preferred embodiments of the invention and should not be considered to limit the scope of the invention.

FIGS. 1A-1B shows heatmaps of the 80-gene expression levels in the training set (UARK2003-33, n=142). FIG. 1A: heatmap of the post-bortezomib 80-gene expression levels, which are mean-centered and scaled for each gene (row). Genes are ordered by results of hierarchical cluster analysis where the average linkage method and the Pearson correlation metric were used. Columns (samples) are ordered by the post-bortezomib 80-gene score (PBS) in ascending order, which is indicated by the green triangle. The horizontal yellow line separates the two major gene clusters, with the upper cluster consisting of numerous genes coding for subunits of the proteasome as indicated by the vertical red bar. The vertical yellow line separates the high and low-risk groups defined by the post-bortezomib 80-gene score. FIG. 1B: heatmap of baseline expression of the 80 genes with columns (samples) and rows (genes) ordered the same way as in the upper panel. Data are mean-centered and scaled for each gene.

FIG. 2 shows statistically significant protein ubiquitination pathway from Ingenuity Pathway Analysis (IPA) of the 80 selected genes where the red filled shapes represent genes upregulated in the high-risk group defined by the post-bortezomib 80-gene score.

FIGS. 3A-3D show the survival analysis in the training set (UARK2003-33). FIG. 3A shows Kaplan-Meier curves of event-free survival (EFS) in high and low-risk groups defined by the post-bortezomib 80-gene score (PBR). FIG. 3B shows Kaplan-Meier curves of OS in high and low-risk groups defined by the post-bortezomib 80-gene score. FIG. 3C shows Kaplan-Meier curves of event-free survival in the four risk groups defined by the baseline 70-gene score and post-bortezomib 80-gene score combined. FIG. 3D shows Kaplan-Meier curves of OS in the four risk groups defined by baseline 70-gene score and post-bortezomib 80-gene score combined. In both FIG. 3C and FIG. 3D, the baseline 70-gene score-low/post-bortezomib 80-gene score-high group appears to have poorer survival than the baseline 70-gene score-high/post-bortezomib 80-gene score-low group although the difference is not significant by the log-rank test.

FIGS. 4A-4B shows heatmaps of the 80-gene expression levels in the test set (UARK2006-66). FIG. 4A: heatmap of 80-gene 48 hr-expression levels in the test set with columns (samples) ordered by the post-bortezomib 80-gene score (PBS) in ascending order and rows (genes) ordered as in the training set of FIG. 1. FIG. 4B: heatmap of baseline 80-gene expression levels with columns (samples) and rows (genes) ordered the same way as in the training set of FIG. 1.

FIG. 5 show distributions of the post-bortezomib 80-gene score in the training and test sets where the red vertical line separates the high and low-risk groups defined by the post-bortezomib 80-gene score at 2.48.

FIGS. 6A-6B show survival analysis in the test set (UARK2006-66). FIG. 6A show Kaplan-Meier curves of event-free survival in predicted high and low-risk groups defined by the post-bortezomib 80-gene score. FIG. 6B show Kaplan-Meier curves of OS in predicted high and low-risk groups defined by the post-bortezomib 80-gene score. FIG. 6C shows Kaplan-Meier curves of event-free survival in the four risk groups defined by baseline 70-gene score and post-bortezomib 80-gene score combined. FIG. 6D shows Kaplan-Meier curves of OS in the four risk groups defined by baseline 70-gene score and PBR combined. For both FIG. 6C and FIG. 6D, the BLR-low/post-bortezomib 80-gene score-high group appears to have poorer survival than the baseline 70-gene score-high/post-bortezomib 80-gene score-low group although the difference is not significant by the log-rank test.

FIG. 7A shows distribution of high and low-risk defined by the post-bortezomib 80-gene score in molecular subgroups in the training and test sets combined (p-value<0.001). FIG. 7B shows distribution of high and low-risk defined by the baseline 70-gene score in molecular subgroups in the training and test sets combined (p-value<0.001).

FIG. 8A-8B shows by mass spectrometry, the effects of bortezomib on proteasome proteins. Representative examples of proteasome up-regulation after bortezomib at both the RNA and protein levels are depicted.

FIG. 9 shows Kaplan-Meier curves by high and low-risk groups defined by baseline 80-gene score in Total Therapy 2 (TT2) (n=351) where 4% was identified as high-risk.

FIG. 10 shows Bar plots of gene expression changes on selected proteasome genes (PSMB2, PSMB3, PSMC5, and PSMD14) after short-term exposure to bortezomib (Bor), dexamethasone (Dex), thalidomide (Thal), and Melphalan (Mel). The figure reveals that the proteasome genes did not change after dexamethasone and thalidomide and, in the case of Melphalan, the changes for some proteasome genes (e.g. PSMB3 and PSMD14) were reversed compared to the changes after bortezomib. This suggests that the proteasome gene up-regulation is unique to bortezomib.

### DETAILED DESCRIPTION OF THE INVENTION

Other and further aspects, features, and advantages of the present invention will be apparent from the description of the presently preferred embodiments of the invention. These embodiments are given for the purpose of disclosure.

The use of the word “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method or composition described herein can be implemented with respect to any other method or composition described herein.

The use of the term “or” in the claims is used to mean “and/or” unless explicitly indicated to refer to alternatives only or the alternatives are mutually exclusive, although the disclosure supports a definition that refers to only alternatives and “and/or.”

In one embodiment of the present invention, there is provided a method for predicting the likelihood of transformation from a low-risk prognosis to a high-risk prognosis for a subject with multiple myeloma, comprising obtaining a gene expression profile of tumor cell genes before administration of a chemotherapeutic agent, administering a single dose of a chemotherapeutic agent to the subject, obtaining a gene expression profile of tumor cell genes after administration of the chemotherapeutic agent and comparing the before and after gene expression profiles, where the upregulation of genes in the profile obtained after administration compared to the profile before administration is indicative of a likelihood of transformation to a high-risk prognosis. In a related embodiment, the genes are selected from the group consisting of COX6C, NOLA1, COPS5, SOD1, TUBA6, HNRPC, PSMB2, PSMC4, LOC400657, C1orf31, FUNDC1, SUMO1, PSMB4, PSMB3, ENSA, PSMB4, COMMD8, MRPL47, PSMC5, PSMA4, PSMD4, NMT1, PSMB7, NXT2, SLC25A14, PSMD2, SNRPD1, CHORDC1, PSMD14, LAP3, PSMA7, UBPH, BIRC5, STAU2, ALDOA, TMC8, C1 orf 128, FLNA, HIST1H3B. The genes may be proteosome genes. The chemotherapeutic agent may be bortezomib. The gene expression may be determined at the nucleic acid or protein level. The gene expression profile after administration of chemotherapeutic agent may be obtained in about 48 hours.

In another related embodiment, the method of predicting the likelihood of transformation may further comprise the step of designing a therapeutic regimen effective to prevent transformation to high-risk state by suppressing the hyperactivation of upregulated genes.

In another related embodiment, the method for predicting the likelihood of transformation further comprises assigning a score based on the correlation of the upregulated genes expression profile to a risk of transformation in the prognosis for the subject. The risk of transformation may be determined using multivariate analyses.

In another embodiment of the presented invention, there is provided a method for identifying latently aggressive multiple myeloma tumor cells in a subject, comprising obtaining a first gene expression profile of multiple myeloma tumor cells in the subject, contacting the tumor cells with a single chemotherapeutic agent and obtaining a second gene expression profile of the multiple myeloma tumor cells after contact with the chemotherapeutic agent, where upregulation of genes in the second profile compared to the first profile is indicative that the tumor cells are latently aggressive tumor cells. The genes are selected from the group consisting of COX6C, NOLA1, COPS5, SOD1, TUBA6, HNRPC, PSMB2, PSMC4, LOC400657, C1orf31, FUNDC1, SUMO1, PSMB4, PSMB3, ENSA, PSMB4, COMMD8, MRPL47, PSMC5, PSMA4, PSMD4, NMT1, PSMB7, NXT2, SLC25A14, PSMD2, SNRPD1, CHORDC1, PSMD14, LAP3, PSMA7, UBPH, BIRC5, STAU2, ALDOA, TMC8, C1orf128, FLNA, HIST1H3B. The tumor cell genes may be proteosome genes. The chemotherapeutic agent may be bortezomib. Gene expression may be determined at the nucleic acid or protein level. The second gene expression profile may be obtained about 48 hours after administration of chemotherapeutic agent.

In a related embodiment, the method for identifying latently aggressive multiple myeloma tumor cells in a subject may further comprise the step of predicting the likelihood that the subject will transform to a high-risk prognosis based on the level of gene activation in the second profile.

In yet another embodiment of the present invention, there is a method for treating multiple myeloma in a subject, comprising determining if the multiple myeloma tumor cells in the subject are latently aggressive, designing a chemotherapeutic regimen comprising one or more anticancer agents effective to suppress activation of genes in the latently aggressive tumor cells and administering the one or more anticancer agents to the subject thereby treating the multiple myeloma.

In a related embodiment, the step of determining if multiple myeloma tumor cells are latently aggressive may comprise obtaining a first gene expression profile of multiple myeloma tumor cells in the subject, contacting the tumor cells with a single chemotherapeutic agent and obtaining a second gene expression profile of the multiple myeloma tumor cells after contact with the chemotherapeutic agent; wherein activation of genes in the second profile compared to the first profile is indicative that the tumor cells are latently aggressive. The genes may be selected from the group consisting of COX6C, NOLA1, COPS5, SOD1, TUBA6, HNRPC, PSMB2, PSMC4, LOC400657, C1orf31, FUNDC1, SUMO1, PSMB4, PSMB3, ENSA, PSMB4, COMMD8, MRPL47, PSMC5, PSMA4, PSMD4, NMT1, PSMB7, NXT2, SLC25A14, PSMD2, SNRPD1, CHORDC1, PSMD14, LAP3, PSMA7, UBPH, BIRC5, STAU2, ALDOA, TMC8, C1orf128, FLNA, HIST1H3B. The tumor cell genes may be proteosome genes. The chemotherapeutic agent may be bortezomib. The gene expression may be determined at the nucleic acid or protein level. The second gene expression profile may be obtained about 48 hours after administration of the chemotherapeutic agent. The anticancer agent may be bortezomib or thalidomide or combination thereof.

In still another embodiment of the present invention, there is a method for predicting the likelihood of transformation from a low-risk prognosis to a high-risk prognosis for a subject with multiple myeloma, comprising obtaining a gene expression profile of tumor cell genes before administration of a chemotherapeutic agent, administering a single dose of a chemotherapeutic agent to the subject, obtaining a gene expression profile of tumor cell genes after administration of the chemotherapeutic agent and comparing the before and after gene expression profiles, where the downregulation of genes in the profile obtained after administration compared to the profile before administration is indicative of a likelihood of transformation to a high-risk prognosis. The genes are selected from a group consisting of FOSB, LOC644250, C17orf60, LZTR2, PDE4B, STAU2, PDE4B, GABARAPL1, TAGAP, LOC643318, CISH, NR4A1, MGC61598, ANKRD37, KIAA1394, ACVR1C, TBCID9, CRYGS, PDE4B, ZNF710, RBM33, STX11, K1AA1754, RPL41, WIRE, LAPTM4A, KLHL7, C9orf130, C14orf100. The chemotherapeutic agent may be bortezomib. Gene expression may be determined at the nucleic acid or protein level. Gene expression profile after administration of chemotherapeutic agent may be obtained in about 48 hours.

In a related embodiment, the method for predicting the likelihood of transformation further comprises the step of assigning a score based on the correlation of the downregulated genes expression profile to a risk of transformation in the prognosis for the subject. The risk of transformation may be determined using multivariate analyses.

In a related embodiment, the method for predicting the likelihood of transformation further comprises the step of designing a therapeutic regimen effective to prevent transformation to the high-risk state by hyperactivating the downregulated genes.

In still another embodiment of the present invention, there is a method for identifying latently aggressive multiple myeloma tumor cells in a subject, comprising obtaining a first gene expression profile of multiple myeloma tumor cells in the subject, contacting the tumor cells with a single chemotherapeutic agent and obtaining a second gene expression profile of the multiple myeloma tumor cells after contact with the chemotherapeutic agent, where downregulation of genes in the second profile compared to the first profile is indicative that the tumor cells are latently aggressive tumor cells. The genes may be selected from the group consisting of FOSB, LOC644250, C17orf60, LZTR2, PDE4B, STAU2, PDE4B, GABARAPL1, TAGAP, LOC643318, CISH, NR4A1, MGC61598, ANKRD37, KIAA1394, ACVR1C, TBCID9, CRYGS, PDE4B, ZNF710, RBM33, STX11, K1AA1754, RPL41, WIRE, LAPTM4A, KLHL7, C9orf130, C14orf100. The chemotherapeutic agent may be bortezomib. The gene expression may be determined at the nucleic acid or protein level. The second gene expression profile may be obtained about 48 hours after administration of the chemotherapeutic agent.

In a related embodiment, the method for identifying latently aggressive multiple myeloma tumor cells in a subject, further comprises the step of predicting the likelihood that the subject will transform to a high-risk prognosis based on the level of gene suppression in the second profile.

In another embodiment of the present invention, there is provided a method for treating multiple myeloma in a subject, comprising determining if the multiple myeloma tumor cells in the subject are latently aggressive, designing a chemotherapeutic regimen comprising one or more anticancer agents effective to activate genes in the latently aggressive tumor cells and administering the one or more anticancer agents to the subject thereby treating the multiple myeloma. The genes may be selected from the group consisting of FOSB, LOC644250, C17orf60, LZTR2, PDE4B, STAU2, PDE4B, GABARAPL1, TAGAP, LOC643318, CISH, NR4A1, MGC61598, ANKRD37, KIAA1394, ACVR1C, TBC1D9, CRYGS, PDE4B, ZNF710, RBM33, STX11, K1AA1754, RPL41, WIRE, LAPTM4A, KLHL7, C9orf130, C14orf100.

In a related embodiment, the step of determining if the multiple myeloma tumor cells are latently aggressive comprises obtaining a first gene expression profile of multiple myeloma tumor cells in the subject, contacting the tumor cells with a single chemotherapeutic agent and obtaining a second gene expression profile of the multiple myeloma tumor cells after contact with the chemotherapeutic agent, wherein suppression of genes in the second profile compared to the first profile is indicative that the tumor cells are latently aggressive. The chemotherapeutic agent may be bortezomib. Gene expression may be determined at the nucleic acid or protein level. The second gene expression profile may be obtained about 48 hours after administration of the chemotherapeutic agent. The anticancer agent may be bortezomib or thalidomide or combination thereof.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### Example 1

### Bortezomib Pharmacogenomics Identify Mechanisms Of Drug Resistance And Predict Survival In Multiple Myeloma Treated With Total Therapy 3

The prognosis of patients with multiple myeloma is best captured by gene expression profiling analysis of CD138-purified plasma cells, distinguishing a high-risk group of 15% with dismal survival using a 70-gene baseline risk model (BLR). Translational research in Total Therapy 3 (TT3) was designed to investigate whether short-term bortezomib-induced gene expression profiling alterations could advance our understanding of bortezomib's novel mechanism of action.

Pharmacogenomic (PG) studies were performed as part of two TT3 trials (2003-33, n=303; 2006-66, n=177), obtaining plasma cells prior to and 48 hr after a bortezomib test-dose (1.0 mg/m2), which was accomplished in 142 patients receiving 2003-33 (training set) and 127 receiving 2006-66 (test set). Among 1051 genes significantly altered post-bortezomib in the training set, 80 were identified as being significantly associated with event-free survival. A continuous risk score was calculated and an optimal cut-point for event-free survival separation determined. The independent prognostic power of the binary risk score was tested in 2006-66. Multivariate analyses (MV) were employed to determine the role of post-bortezomib risk in relationship to standard prognostic variables and 70-gene baseline risk model.

The discriminatory power in 2003-33 (3-yr OS: 95% v 45%, p<0.0001; 3-yr event-free survival: 90% v 35%, p<0.0001) was confirmed in 2006-66 (18-mo OS: 100% v 65%, p=0.0004; 18-mo event-free survival: 95% v 45%, p<0.0001). Evaluating PBR in the context of 70-gene baseline risk model, 12/26 in 2003-33 and 7/21 in 2006-66 deemed as having low 70-gene baseline risk had a high post-bortezomib 80-gene binary score. Conversely, 8/126 in 2003-33 and 14/106 in 2006-66 deemed as having high 70-gene baseline risk had low post-bortezomib 80-gene binary score. In the context of the 8 molecular subgroup model, high post-bortezomib 80-gene binary score was over-represented in the Proliferation (PR) subgroup (7/15 in 2003-33, 8/18 in 2006-66) and absent in the Low Bone disease (LB) group (0/28). On multivariate analysis, the post-bortezomib 80-gene binary score was an independent adverse variable for both OS and event-free survival in 2003-33 (OS: HR=3.17, p=0.006, R2=55%; event-free survival: HR=4.40, p<0.001, R2=48%) and in 2006-66 (OS: HR=13.00, p=0.002, R2=48%; event-free survival: HR=15.57, p<0.001, R2=55%). Therefore, pharmacogenomic research has identified a powerful 80-gene PBR model with unprecedented prognosis-discriminating power, dispelling 70-gene-derived BLR from multivariate analysis by altering BLR designation mainly from low to high risk. High PBR (18%) could be traced to up-regulation of proteasome genes, the target of bortezomib.

Given that PSMD4 maps to the 1q21 amplicon and its elevated expression is associated with increased risk in Total Therapy 2 (TT2), PSMD4 GEP might represent a useful biomarker to identify patients who may benefit from the increased efficacies seen with the use of bortezomib in induction, consolidation, and maintenance in Total Therapy 3 (TT3). Furthermore, based on observations in childhood acute lymphoblastic leukemia and myeloma, an unbiased read-out of a resistance-associated genomic signature could be revealed by a test-dose administration of bortezomib in vivo.

To examine this issue and to search for a molecular basis of mechanism of action of bortezomib, genome-wise mRNA expression profiling of purified CD138-selected plasma cells was employed prior to and following a short-term test dose of bortezomib. The objective, similar to examinations of Thalidomide, Dexamethasone, and Lenalidomide was to identify, in a global unbiased fashion allowing all genes to compete in a log rank test, genes whose expression was altered by the drug and whose post drug expression level was related to outcome. A total of 80 genes were identified whose expression changed following 48 hr exposure to bortezomib and the post-drug expression level was related to outcome. This list contained a significant overrepresentation of proteasome genes, including PSMD4, whose expression increased following the test dose.

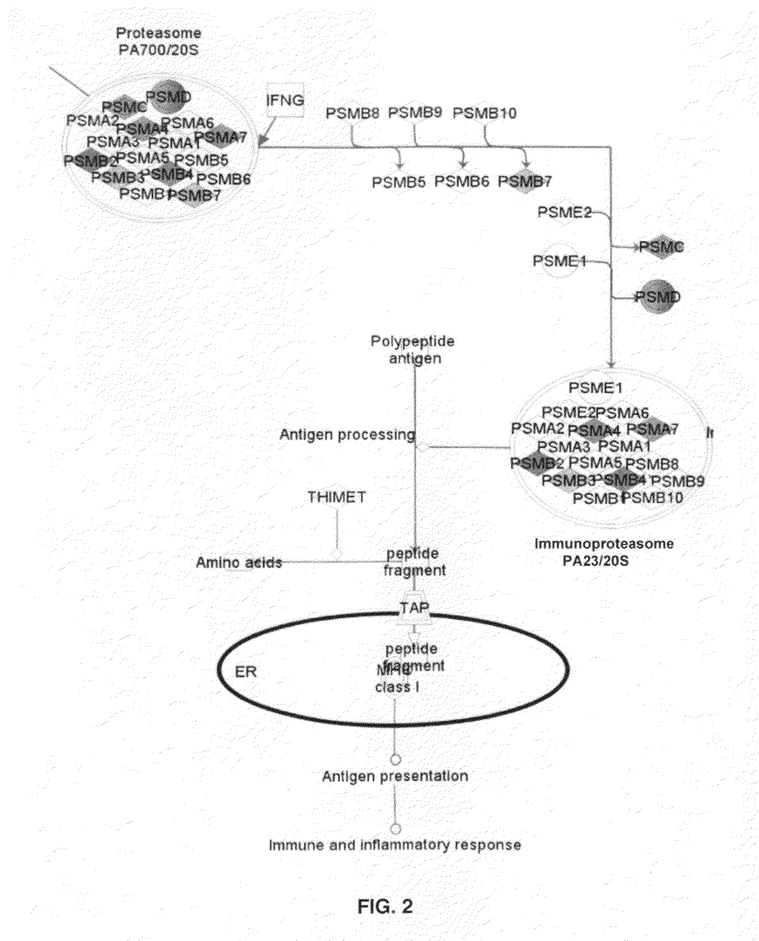

Consistently, Ingenuity Pathway Analysis (FIG. 2) revealed a significant association of this 80-gene list with the proteasome pathway. Importantly, short-term thalidomide, dexamethasone, or lenalidomide (Burington et al., 2009) was not associated with hyperactivation of proteasome genes, suggesting this phenomenon was bortezomib specific. The 80-gene model was validated in separate cohort of newly diagnosed disease treated with the same therapy (TT3B). The model was also able to predict outcome in baseline TT3 and TT3B samples, indicating that elevated expression of proteasome genes in therapy naïve disease was also related to outcome in the bortezomib containing TT3/T3B regimen.

To determine if the 80-gene model provided additional prognostic information above that achieved with the 70-gene model, Kaplan Meier analyses was performed following high- verses low-risk prediction by the 70-gene and 80-gene models. These studies revealed that in TT3 and TT3B, a significant fraction of cases deemed to be high risk by the 70-gene model but low risk by the 80-gene model enjoyed a significantly improved outcome relative to cases deemed high risk by both models, yet inferior to those for whom their disease was predicted to be low risk by both models.

This phenomenon, however, was not observed in patients treated with TT2. Specifically, cases predicted to be low risk by the 70-gene model and low risk by the 80-gene model, experienced similar outcomes to those who were high risk by both models. Additionally, for patients who were predicted to be low risk by both models their superior outcome were similar with TT2 and TT3. Likewise, those with high-risk disease by both models had equally poor outcomes. It is noteworthy that the improved survival of those with 70-gene high risk/80-gene low risk disease was still inferior to those with low risk by both models. These data imply that TT3 improved the outcome in a subset of high-risk disease as defined by the 70-gene model and these cases have proteasome activation that is intermediate to the high levels in high risk and low levels in low risk disease.

With this result in mind, it could be considered that outcome might be modeled by a graded expression of the PSMD4 gene and that this graded expression was related to copy number gains of the PSMD4 locus. To this end, interphase FISH for 1q21 copy number gains and gene expression profiling was performed in 600 cases of newly diagnosed multiple myeloma and were able to demonstrate that PSMD4 mRNA levels, as well as those of IL6R, CKS1B, and MCL1, but not BCL9, was progressively higher as copy number of the 1q21 locus increased from 2 to 3 to 4 or more copies of 1q21. An optimal cut point of PSMD4 gene expression profiling corresponding to 2, 3, and 4+ copies of 1q21 identified and these cut points related to outcome in TT2 and TT3. The results revealed that cases with PSMD4 gene expression profiling in the lowest tertile fared equally well with TT2 and TT3.

As expected, patients with PSMD4 in the middle tertile experienced a significant improvement with TT3 relative the same group treated with TT2, and PSMD4 expression in the highest tertile was associated with a dismal prognosis in both TT2 and TT3. Importantly, a significant number of cases with 70-gene high-risk/80-gene low risk had PSMD4 in the middle tertile. Given that three molecular variables, PSMD4 gene expression cut-points, FISH for 2, 3, and 4+ copies of 1q21, and the combined 70/80-gene models, appeared to be defining a common biological phenomenon related to outcome in multiple myeloma, i.e. variation in copy number of the 1q21 locus, the relative prognostic impact of all three variables was evaluated in a multivariate Cox regression analysis with standard variables.

The results revealed that the combined 70/80-gene model retained prognostic independence. Importantly, it was also independent of the now recognized improved outcome in TT3 of disease with delTp53 and the MS subtype. R² statistics also revealed that the combined 70/80-gene model alone could account for an unprecedented 50% of outcome variability in the TT3 trials. These data point to the incredible power of gene expression profiling to provide a means of disease classification, prognostication and prediction, and as revealed here, the potential discovery of mechanisms of action. The data revealed that elevated expression of proteasome, but not the immmunoproteasome, genes in multiple myeloma tumor cells exposed through pharmacogenomic studies with an in vivo challenge with a therapeutic dose of bortezomib, that the improved outcome associated with TT3 relative to TT2, and thus the benefits of the use of bortezomib throughout the course of therapy, can be traced variability in the expression of the proteasome gene PSMD4, a copy number sensitive gene mapping to the 1q21 amplicon in this disease. In fact, these data revealed that the inclusion of bortezomib+THAL at the doses and schedule used in TT3 improved the outcome of disease with moderate expression of PSMD4 corresponding to trisomy of 1q21, while cases with the highest level of PSMD4, corresponding to four or more copies of the 1q21 locus failed to show an improvement. Together these data suggest that the relative proteasome activity in multiple myeloma cells, perhaps driven by increased copy number and activity of PSMD4, may determine the relative sensitivity of tumor cells to apoptosis and effective tumor cell eradication by bortezomib+THAL in combination with poly-chemotherapy. Novel therapeutic strategies aimed at overcoming the effects of the hyperactivated proteasome may be central to overcoming high-risk in multiple myeloma.

### Patients and Methods

The details of Total Therapy 3 are known in the art. In brief, protocol 2003-33 enrolled 303 newly diagnosed patients with myeloma into a tandem transplantation program, employing 2 cycles each of induction chemotherapy with VTD-PACE (bortezomib, thalidomide, dexamethasone; 4-day continuous infusions of cisplatin, doxorubicine, cyclophosphamide, etoposide) prior to and after melphalan-based transplantation, followed by VTD maintenance. A pharmaco-genomic aspect of the protocol called for gene expression profiling analyses of CD138-purified plasma cells obtained prior to and 48 hr following a test-dose application of bortezomib (1.0 mg/m2). The observation of prognostic consequences of bortezomib-induced gene expression profiling alterations provided the rationale for accrual of an additional 177 patients to an extension TT3 protocol, 2006-66, in order to validate the pharmaco-genomic data generated in the 2003-33 trial. Gene expression profiling baseline data were available in 275 and 169 patients entered on 2003-33 and 2006-66, respectively, 142 and 128 of whom had paired samples at baseline and 48 hr after test-dosing.

Both studies had been approved by the institutional review board, and patients provided written informed consent acknowledging the investigational nature of the protocols. The records of at least 80% of enrolled patients had been audited for protocol adherence as well as toxicity and efficacy data by a federally accredited team of investigators.

### Gene Expression Profiling

Multiple myeloma plasma cells were purified from bone marrow aspirates prior to and 48 hr following in vivo exposure to 1.0 mg/m2 of bortezomib in 142 of 303 patients enrolled in UARK2003-33 and 128 of 177 accrued to UARK2006-66. RNA was isolated from tumor cells and run on Affymetrix U133Plus2.0 microarrays. Multiple myeloma plasma cells were isolated from heparinized bone marrow (BM) aspirates using CD138-based immunomagnetic bead selection using the Miltenyi AutoMacs device (Miltenyi, Bergisch Gladbach, Germany). Peripheral Blood mononuclear cells were obtained through Ficoll gradient centrifugation. RNA purification, cDNA synthesis, cRNA preparation, and hybridization to the Human Genome U133Plus 2.0 GeneChip microarrays (Affymetrix, Santa Clara, Calif.) were performed.

### Statistical Methods

The Affymetrix U133Plus2.0 microarrays were preprocessed and normalized with MAS5.0 software. Using the UARK2003-33 protocol data as a training set (n=142), 1051 genes were identified whose expression levels significantly changed 48 hours after bortezomib relative to baseline by applying a t-test on each gene and a 0.005 p-value cutoff with a false discovery rate of 0.22. Each of these 1051 genes was then correlated with event-free survival in a Cox regression model and ranked the genes by p-values. In order to explore the various potential survival implications, two sets of covariates were considered in the Cox model for each gene: a) percent change at 48 hr compared to the baseline expression, and b) 48 hr expression.

Fitting model (a) on each of the 1051 genes produced a minimum q value of 0.94, which means that even if one selects only the most significant gene, the chance of it being a false positive is 94%. Fitting model (b) produced much smaller q values than model (a) with a minimum of 0.005. This reveals that the 48 hr expression, compared with the percent change, was more associated with survival. Accordingly, model (b) was chosen over model (a) and ranked genes by the p values of the 48 hr expression.

Once genes are ranked, one can predict survival by selecting the top x number of genes and computing a summary score for each patient as described in Shaughnessy et al (2007). The score for a particular patient is defined as the mean differential expression of good genes and bad genes, where good genes are those associated with favorable outcome (hazard ratio or HR<1) and bad genes unfavorable outcome (HR>=1). The score is computed in such a way that the higher the score the higher the risk of having an event.

To characterize patients into high and low-risk groups one can dichotomize the continuous summary score by finding an optimal cut-point to maximize the survival difference between the resulting two groups. Patients having scores greater than the cut-point would be assigned to the high-risk group and patients below to the low-risk group. To produce a robust model, a 10-fold cross-validation approach (Table 1) was utilized to simultaneously select 80 genes to calculate a gene score and an optimal cut-point to dichotomize the score. Once model selection was completed, the independent prognostic power of the post-bortezomib 80-gene score was then examined in multivariate analysis of event-free survival and OS along with other prognostic factors. This score was lastly tested in the test set of the UARK 2006-66 protocol (n=128).

TABLE 1
Divide the training set randomly into 10 parts of approximately equal size.
Let m denote the number of most significant genes to calculate a gene score
and q the q-th percentile to dichotomize the score.
Given (m, q), where m and q are in reasonable ranges, we repeat the following steps:

| | |
|---|---|
| (1) | Train the model with 9 parts of the training set: first select differentially expressed genes with |
| | a 0.005 p-value cutoff, followed by ordering the selected genes by univariate association |
| | with survival, then calculate a gene score as in [] based on the top m genes and calculate |
| | the q-th percentile of this score, which we denote by w. |
| (2) | Test the model with the remaining part: calculate gene scores using the top m genes |
| | identified in step 1 and dichotomize the scores at w, which is also from step 1. |
| (3) | Repeat step 1-2 10 times. Every sample in the training set is now assigned a label of 0 or 1. |
| | Compute the log rank test between group 0 and 1 and record the p-value. |
| | Select the best combination of (m, q) that gives the lowest p-value. |

Alterations of Gene Expression 48 Hr after Bortezomib Test-Dosing

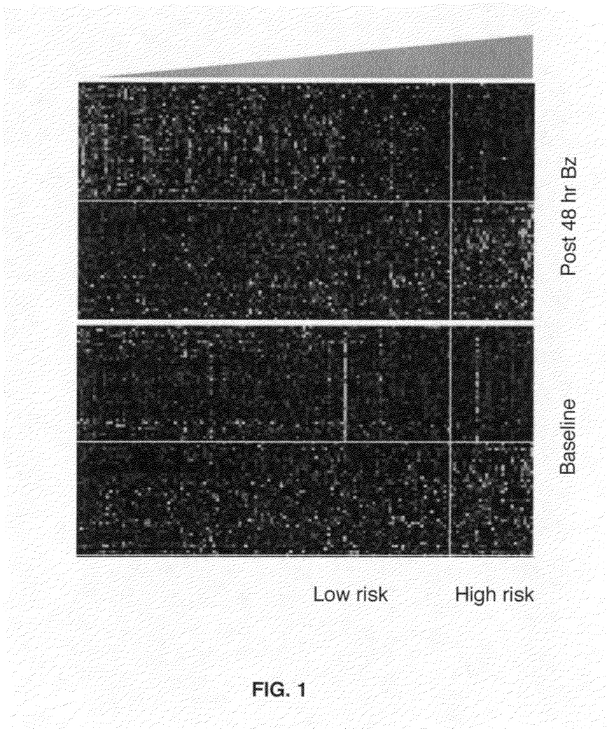

Using the training set of 142 patients in trial 2003-33, 80 differentially expressed genes was identified (Table 2) whose 48 hr expression levels were associated with event-free survival. FIG. 1A shows a heatmap of the 80-gene expression levels at 48 hr, centered and scaled for each gene. The heatmap reveals two major gene clusters with the upper gene cluster consisting of numerous genes coding for subunits of the proteasome. The high-risk group characterizes the concerted up-regulation of 42 bad genes (including genes coding for the proteasome) and down-regulation of 38 good genes. Applying Ingenuity Pathway Analysis, it was verified the proteasome pathway was dominantly affected whereby the assembly of the immunoproteasome was inhibited and proteasome subunits preferentially hyper-activated (FIG. 2).

TABLE 2
Post-bortezomib 80 genes ordered by hazard ratio (HR) regarding 48 hr expression

| | | | | EFS HR | |
|---|---|---|---|---|---|
| | | | EFS | re | Mean |
| | Gene | Chromosomal | p-value re 48 | 48 hr | log2 Expressi [symbol] |
| Probeset | Symbol | Location | expression | expression | Change |
| Bad Genes | | | | | |
| 201754_at | COX6C | chr8q22-q23 | 0.00027 | 4.4 | 0.12 |
| 219110_at | NOLA1 | Chr4q25 | 0.0014 | 3.4 | 0.11 |
| 201652_at | COPS5 | chr8q13.2 | 0.00093 | 3.2 | 0.12 |
| 200642_at | SOD1 | chr21q22.1\|21q22.11 | 0.0014 | 3.1 | 0.15 |
| 209251_x_at | TUBA6 | chr12q12-q14 | 0.0017 | 3.0 | 0.12 |
| 200014_s_at | HNRPC | chr14q11.2 | 0.012 | 2.9 | 0.11 |
| 200039_s_at | PSMB2 | chr1p34.2 | 0.0037 | 2.8 | 0.17 |
| 201252_at | PSMC4 | chr19q13.11-q13.13 | 0.0082 | 2.7 | 0.16 |
| 226924_at | LOC400657 | chr18q22.3 | 0.0043 | 2.6 | 0.11 |
| 225638_at | C1orf31 | chr1q42.2 | 0.00022 | 2.6 | 0.18 |
| 235346_at | FUNDC1 | chrXp11.3 | 0.0037 | 2.6 | 0.13 |
| 211069_s_at | SUMO1 | Chr2q33 | 0.012 | 2.6 | 0.10 |
| 202244_at | PSMB4 | Chr1q21 | 0.016 | 2.5 | 0.15 |
| 201400_at | PSMB3 | chr17q12 | 0.0081 | 2.5 | 0.13 |
| 202596_at | ENSA | chr1q21.2 | 0.0091 | 2.4 | 0.12 |
| 202243_s_at | PSMB4 | Chr1q21 | 0.0098 | 2.4 | 0.19 |
| 218351_at | COMMD8 | Chr4p12 | 0.0044 | 2.4 | 0.16 |
| 223480_s_at | MRPL47 | chr3q26.33 | 0.022 | 2.4 | 0.09 |
| 209503_s_at | PSMC5 | chr17q23-q25 | 0.0062 | 2.3 | 0.17 |
| 212626_x_at | HNRPC | chr14q11.2 | 0.019 | 2.3 | 0.16 |
| 203396_at | PSMA4 | chr15q25.1 | 0.013 | 2.3 | 0.14 |
| 200882_s_at | PSMD4 | chr1q21.2 | 0.00046 | 2.3 | 0.19 |
| 201157_s_at | NMT1 | chr17q21.31 | 0.016 | 2.3 | 0.12 |
| 200786_at | PSMB7 | chr9q34.11-q34.12 | 0.0083 | 2.3 | 0.15 |
| 209628_at | NXT2 | chrXq22.3 | 8.30E−05 | 2.2 | 0.13 |
| 204587_at | SLC25A14 | chrXq24 | 0.0049 | 2.2 | −0.10 |
| 210460_s_at | PSMD4 | chr1q21.2 | 0.0012 | 2.2 | 0.19 |
| 200830_at | PSMD2 | chr3q27.1 | 0.011 | 2.2 | 0.13 |
| 202690_s_at | SNRPD1 | chr18q11.2 | 0.024 | 2.2 | 0.11 |
| 211609_x_at | PSMD4 | chr1q21.2 | 0.0018 | 2.2 | 0.20 |
| 218566_s_at | CHORDC1 | chr11q14.3 | 0.024 | 2.1 | 0.15 |
| 212296_at | PSMD14 | chr2q24.2 | 0.015 | 2.1 | 0.23 |
| 217933_s_at | LAP3 | chr4p15.32 | 0.0079 | 2.0 | 0.15 |
| 201114_x_at | PSMA7 | chr20q13.33 | 0.023 | 1.9 | 0.21 |
| 205687_at | UBPH | chr16p12 | 0.014 | 1.8 | 0.16 |
| 210334_x_at | BIRC5 | chr17q25 | 0.00029 | 1.8 | −0.35 |
| 229417_at | STAU2 | — | 0.0044 | 1.7 | 0.22 |
| 238996_x_at | ALDOA | chr16q22-q24 | 0.02 | 1.7 | 0.36 |
| 236554_x_at | TMC8 | chr17q25.3 | 0.0015 | 1.7 | 0.24 |
| 229856_s_at | C1orf128 | chr1p36.11 | 0.022 | 1.6 | −0.32 |
| 214752_x_at | FLNA | chrXq28 | 0.01 | 1.5 | 0.18 |
| 208576_s_at | HIST1H3B | chr6p21.3 | 0.018 | 1.5 | 0.40 |
| Average | | | | | 0.13 |
| Good Genes | | | | | |
| 202768_at | FOSB | chr19q13.32 | 0.025 | 0.8 | −0.54 |
| 230292_at | LOC644250 | — | 0.012 | 0.8 | −0.34 |
| 217513_at | C17orf60 | chr17q23.3 | 0.022 | 0.8 | 0.32 |
| 1564423_a_at | LZTR2 | chr1q25.2 | 0.019 | 0.7 | −0.23 |
| 211302_s_at | PDE4B | Chr1p31 | 0.018 | 0.7 | −0.28 |
| 233909_at | STAU2 | — | 0.023 | 0.7 | −0.40 |
| 215671_at | PDE4B | Chr1p31 | 0.0023 | 0.7 | −0.42 |
| 208869_s_at | GABARAPL1 | chr12p13.2 | 0.023 | 0.7 | 0.20 |
| 1552542_s_at | TAGAP | chr6q25.3 | 0.0093 | 0.7 | −0.19 |
| 1561060_at | — | — | 0.017 | 0.7 | −0.27 |
| 1557633_at | LOC643318 | chr22q11.2 | 0.025 | 0.7 | −0.29 |
| 223961_s_at | CISH | chr3p21.3 | 0.00015 | 0.7 | −0.32 |
| 202340_x_at | NR4A1 | chr12q13 | 0.00027 | 0.7 | −0.79 |
| 226499_at | MGC61598 | chr9q34.3 | 0.017 | 0.7 | −0.29 |
| 242548_x_at | ANKRD37 | chr4q35.1 | 0.022 | 0.7 | −0.39 |
| 228984_at | K1AA1394 | chr11q13.1 | 0.0049 | 0.6 | −0.29 |
| 229388_at | — | — | 0.012 | 0.6 | 0.22 |
| 1552519_at | ACVR1C | chr2q24.1 | 0.0018 | 0.6 | −0.17 |
| 239343_at | | chr12q23.3 | 0.008 | 0.6 | 0.19 |
| 212960_at | TBC1D9 | chr4q31.21 | 0.0083 | 0.6 | −0.19 |
| 236986_at | — | — | 0.0025 | 0.6 | −0.13 |
| 223643_at | CRYGS | chr3q25-qter | 0.023 | 0.6 | −0.18 |
| 203708_at | PDE4B | Chr1p31 | 0.0018 | 0.6 | −0.25 |
| 221223_x_at | CISH | chr3p21.3 | 7.20E−06 | 0.5 | −0.20 |
| 213658_at | ZNF710 | — | 0.0097 | 0.5 | −0.12 |
| 238801_at | RBM33 | chr7q36.3 | 0.004 | 0.5 | −0.26 |
| 235670_at | STX11 | — | 0.018 | 0.5 | −0.14 |
| 225582_at | K1AA1754 | chr10q25.1 | 0.0074 | 0.5 | −0.14 |
| 227524_at | — | — | 0.01 | 0.5 | −0.17 |
| 239082_at | — | — | 0.0016 | 0.5 | −0.11 |
| 223377_x_at | CISH | chr3p21.3 | 0.00024 | 0.5 | −0.18 |
| 216215_s_at | RPL41 | chr22q13.1 | 0.00013 | 0.5 | −0.16 |
| 212050_at | WIRE | chr17q21.2 | 0.0079 | 0.5 | −0.10 |
| 200673_at | LAPTM4A | chr2p24.1 | 0.021 | 0.4 | 0.12 |
| 223250_at | KLHL7 | chr7p15.3 | 0.023 | 0.4 | 0.09 |
| 227893_at | C9orf130 | chr9q22.32 | 0.018 | 0.4 | 0.16 |
| 223215_s_at | C14orf100 | chr14q23.1 | 0.013 | 0.4 | 0.12 |
| 226399_at | — | — | 0.0092 | 0.4 | 0.09 |
| Average | | | | | −0.16 |

[symbol]

indicates data missing or illegible when filed

### Post-Bortezomib Risk Model Affects Survival Outcomes in Uark2003-33

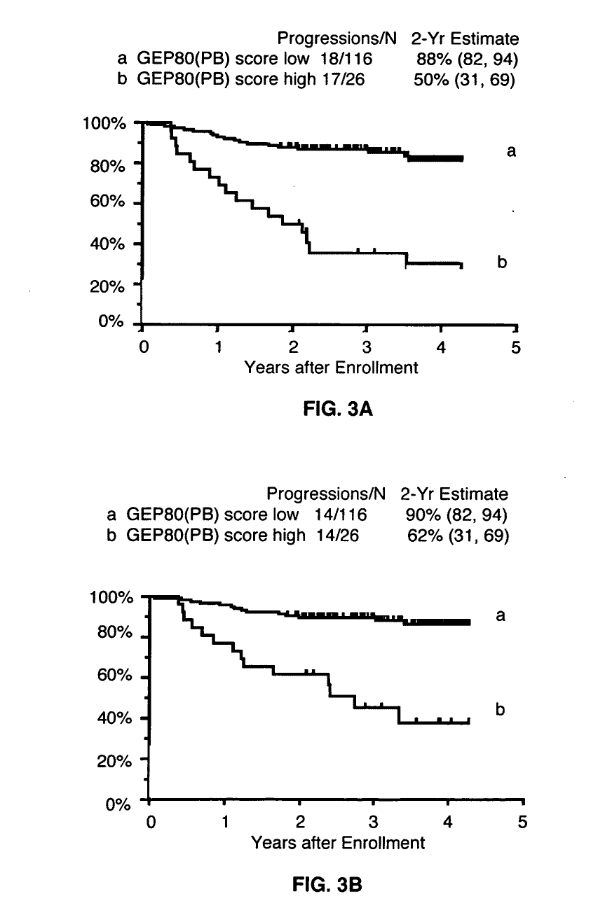

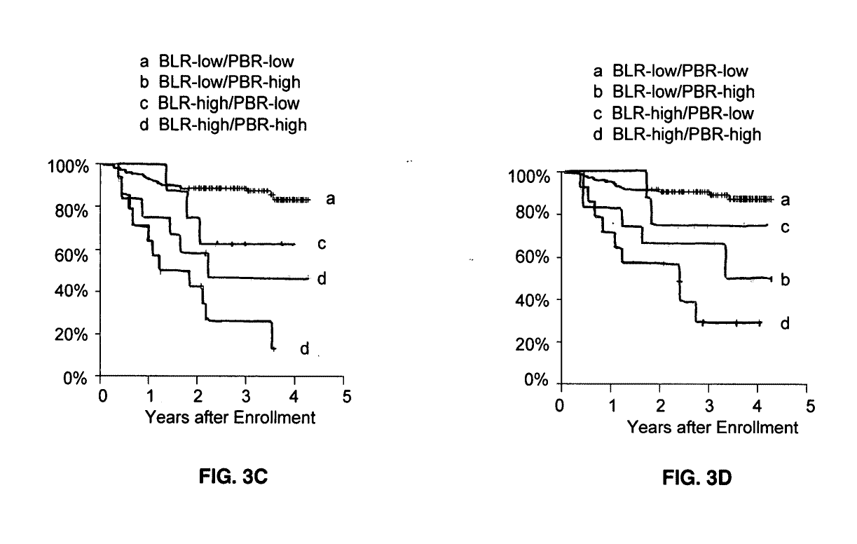

Of the 80 genes linked to event-free survival, 38 were associated with favorable outcome (HR<1.0) and 42 with unfavorable outcome (HR>=1.0). A summary score was calculated based on the 80-gene 48 hr-expression levels and a dichotomizing optimal cut-point of 2.48 defined. Patients having 80-gene scores greater than 2.48 were assigned high-risk and the remainder low-risk designations. Both event-free survival and OS were significantly inferior among the 26 high-risk patients compared to the 116 low-risk patients (FIGS. 3A-3B).

When examining the post-bortezomib 80-gene binary score (PBR) in the context of the 70-gene baseline score (BLR), it was determined that PBR was highly associated with BLR (p<0.001) although only three genes (BIRC5, PSMD4, and K1AA1754) were common to both models. Pertinent to clinical outcome, patients with BLR-low/PBR-low exhibited superior survival, while those with BLR-high/PBR-high had the worst outcomes (FIGS. 3C-3D). The BLR implications were modified adversely by PBR-high for patients with BLR-low features and vice versa, i.e. those with BLR-high were significantly uplifted in their prognosis when PBR was low.

### Validation of Post-Bortezomib 80-Gene Model in Uark2006-66

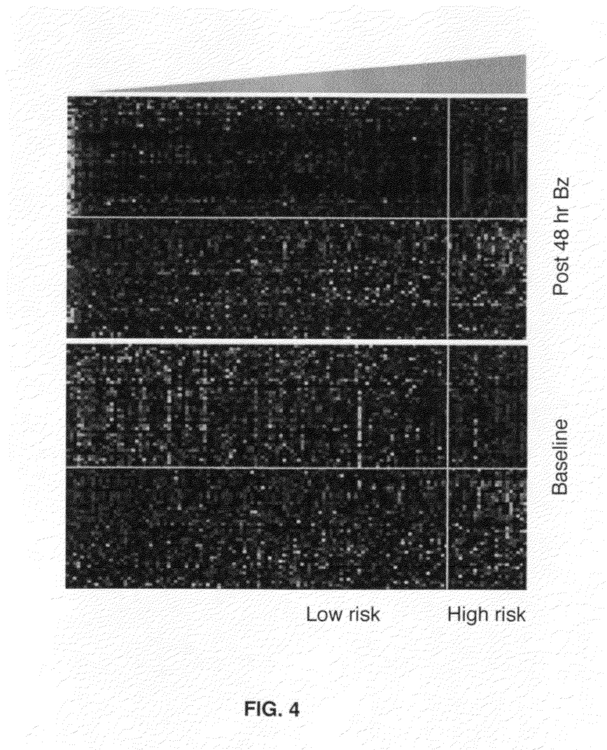

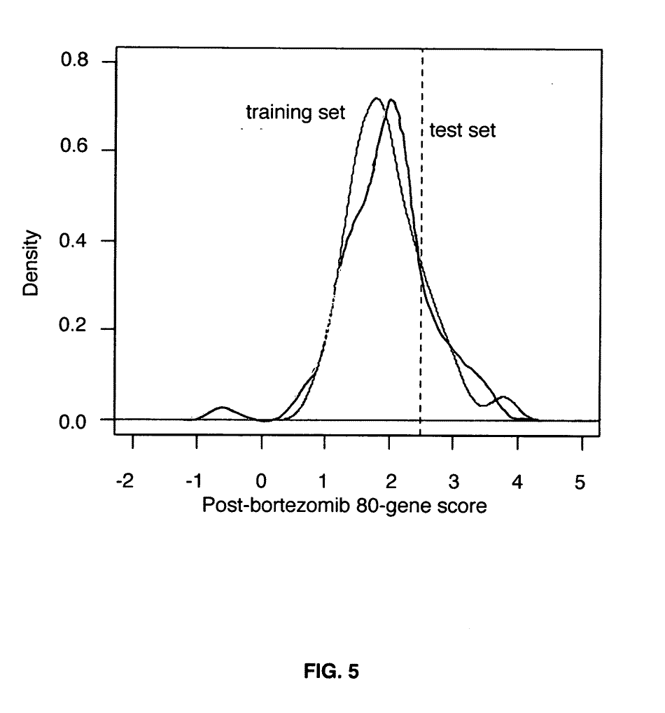

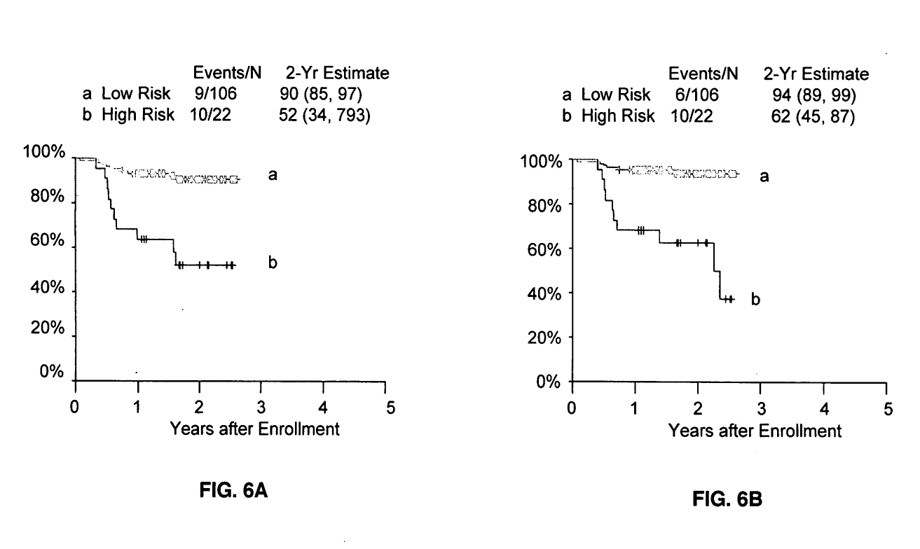

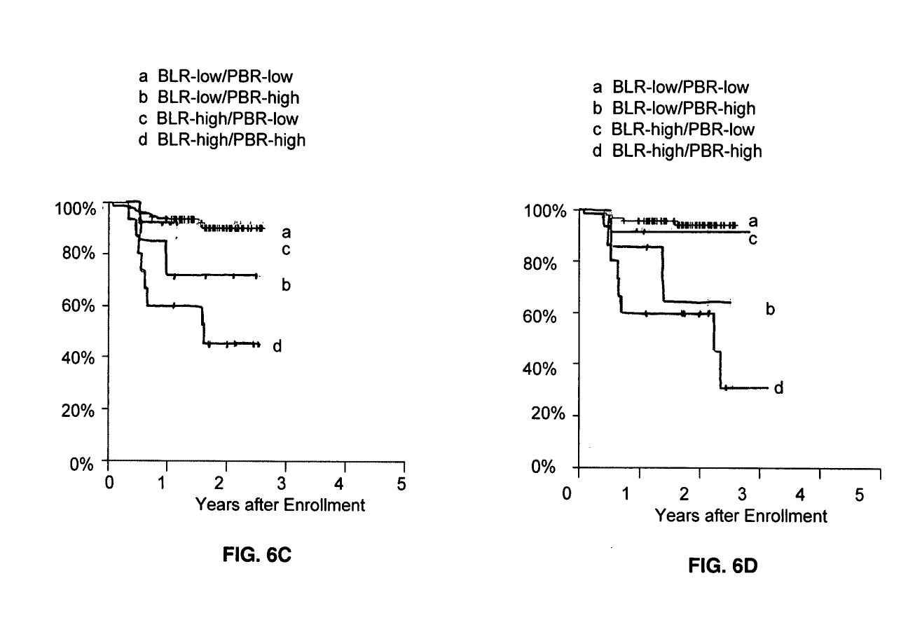

As depicted in FIGS. 4A-4B, the baseline and post-bortezomib 80-gene expression levels in the test set show high similarities to those in the training set of FIGS. 1A-1B. The 80-gene-derived risk score distribution in the test and training sets were almost super-imposable (FIG. 5), providing for a further validation of the post-bortezomib 80-gene model. Indeed, both event-free survival and OS were significantly inferior among the 21 patients with 80-gene-defined high-risk myeloma (FIGS. 6A-6B), providing additional risk definition (as in the training set) when examined together with the 70-gene-based model (FIGS. 6C-6D).

### Post-Bortezomib 80-Gene-Derived High-Risk Distribution Among Myeloma Molecular Subtypes

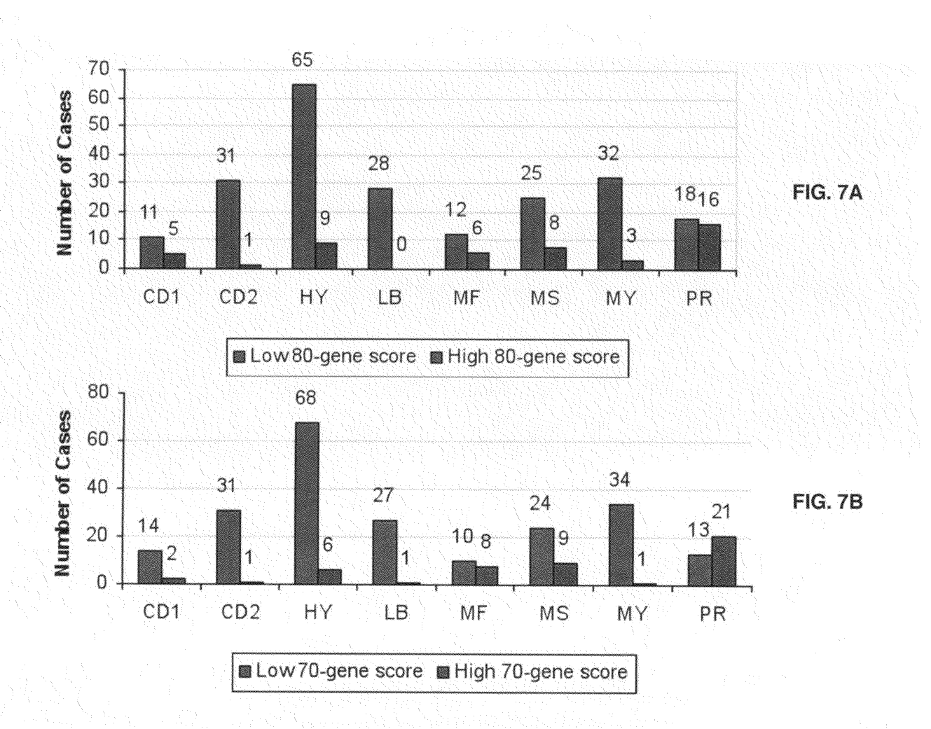

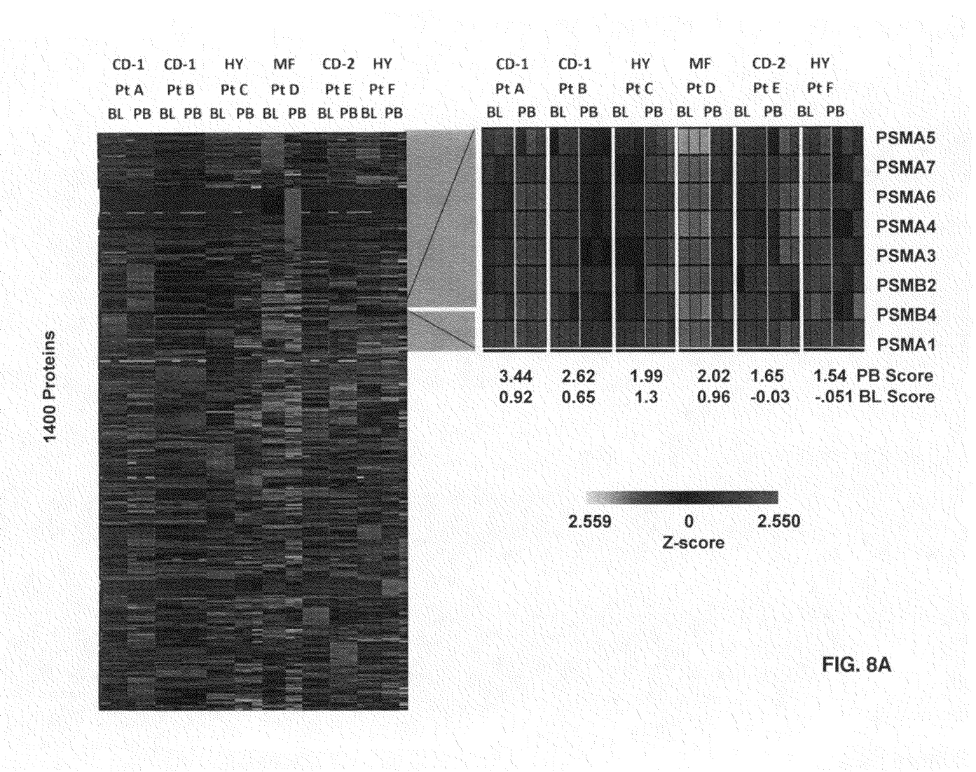

Applying this analysis across both TT3 protocols revealed an under-representation of high-risk in CD-2 and LB subtypes, and over-representation among PR (16 of 34), MF (6 of 18), and MS subtypes (8 of 33) (FIG. 7A). Similar results had been observed for the baseline 70-gene score (FIG. 7B).

### Multivariate Analysis

The prognostic implications of the post-bortezomib 80-gene score in multivariate Cox regression analysis was examined next. While univariately highly significant for both event-free survival and OS, the adverse implications associated with metaphase cytogenetic abnormalities (CA) and 70-gene-derived high-risk no longer imparted poor outcomes when adjusted for the post-bortezomib 80-gene score. Table 3 gives the results from the test set, where the post-bortezomib 80-gene score and delTP53 were selected in the step-wise regression analysis, indicating the independent and superior prognostic power of the 80-gene score.

### Proteasome Proteins Also Increase Post-Bortezomib

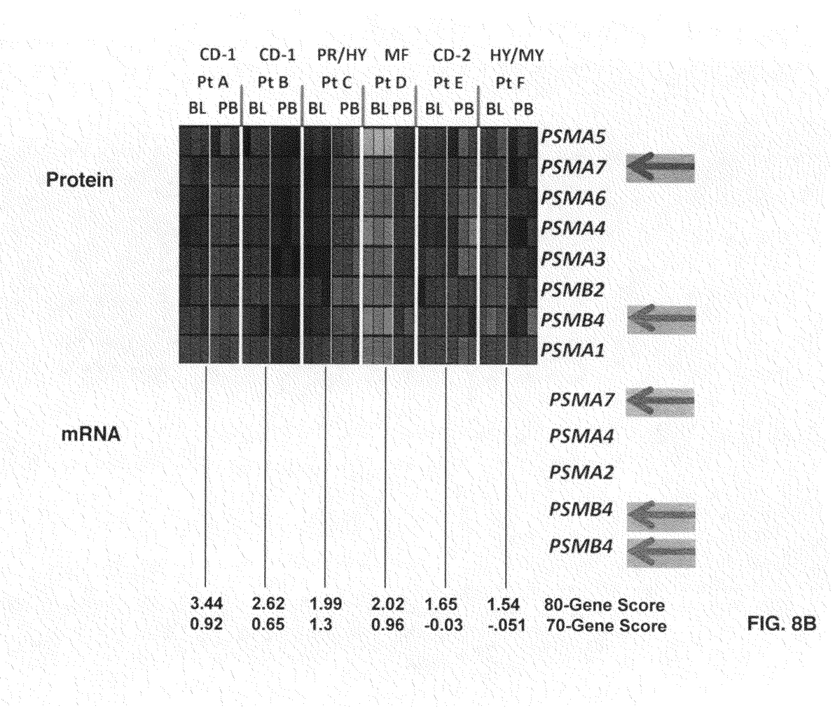

For further validation of GEP-derived data, the effects of bortezomib on proteasome proteins was also investigated by mass spectroscopy, (FIG. 8A). Representative examples of proteasome up-regulation after bortezomib at both the RNA and protein levels are depicted in FIG. 8A-8B.

### Magnitude of Change and Baseline 80-Gene Expression

Knowing that the post-bortezomib 80-gene-defined high-risk group is characterized by up-regulation of the “bad” genes and down-regulation of the “good” genes (FIGS. 1A-1B), the expression change from baseline to post-bortezomib was further examined for each gene. The analysis showed that, on average, the expression in low- and high-risk groups of the bad genes increased by 1.07 and 1.21 fold (p-value<0.0001), and of the good genes decreased by 1.09 and 1.22 fold (p-value=0.03), respectively. Clearly the high-risk group experienced more change than the low-risk group, although the overall change on each individual gene was not prominent (Table 2).

The high similarity in the 80 genes at baseline and 48 hr after bortezomib suggested that the baseline 80-gene score may be similarly predictive of survival as the post-bortezomib 80-gene score. This predictive power of the baseline 80-gene score was confirmed in both the training and test sets with p-value<0.0001 for both event-free survival and OS. As a measure of the level that clinical outcome variability can be accounted for, R² values were computed for both baseline and post-bortezomib 80-gene scores in the test set, yielding values of 25% and 36% for event-free survival, and 32% and 49% for OS, respectively. Importantly, in the absence of the post-bortezomib 80-gene score, the baseline 80-gene score displaced the baseline 70-gene score from the regression model in both training and test sets. When the baseline and post-bortezomib 80-gene scores were both in the model, however, only the post-bortezomib 80-gene score was selected (Table 3). Taken together, these data suggest that, even though the 48 hr-post-bortezomib average-fold change of the 80-gene expression was relatively small, the post-bortezomib 80-gene score is indeed a more powerful predictor than the baseline 80-gene score in capturing outcome variation.

TABLE 3
Multivariate Cox Regression Analysis in the test set (UARK2006-66)

| | | | EFS | | |
|---|---|---|---|---|---|
| | Significant | | | | Cumul. |
| | Predictors | n/N (%) | HR (95% Cl) | P-value | R² (%) |
| Univariate | B2M >= 3.5 mg/L | 70/125 (56%) | 3.47 (1.15, 10.47) | 0.027 | |
| | LDH >= 190 U/L | 32/127 (25%) | 2.98 (1.21, 7.33) | 0.018 | |
| | Cytogenetic Abnormalities | 49/126 (39%) | 2.82 (1.11, 7.17) | 0.029 | |
| | Baseline 70-gene high risk | 27/127 (21%) | 3.82 (1.55, 9.41) | 0.004 | |
| | GEP TP53 deletion | 12/128 (9%) | 4.62 (1.66, 12.84) | 0.003 | |
| | Proliferation index high | 14/127 (11%) | 4.39 (1.67, 11.56) | 0.003 | |
| | Baseline 80-gene high risk | 15/128 (12%) | 5.27 (2.07, 13.41) | <.001 | |
| | Post bortezomib 80-gene | 22/128 (17%) | 6.23 (2.53, 15.34) | <.001 | |
| | high risk | | | | |
| Multivariate | GEP TP53 deletion | 11/122 (9%) | 3.31 (1.15, 9.57) | 0.027 | 19 |
| | Post bortezomib 80-gene | 22/122 (18%) | 4.84 (1.91, 12.29) | <.001 | 38 |
| | high risk | | | | |
| | | | OS | | |
| | Significant | | | | Cumul. |
| | Predictors | n/N (%) | HR (95% Cl) | P-value | R² (%) |
| Univariate | Albumin < 3.5 g/dL | 59/127 (46%) | 2.88 (1.00, 8.30) | 0.050 | |
| | B2M >= 3.5 mg/L | 70/125 (56%) | 3.91 (1.11, 13.73) | 0.034 | |
| | Hb < 10 g/dL | 39/127 (31%) | 2.46 (0.92, 6.56) | 0.072 | |
| | LDH >= 190 U/L | 32/127 (25%) | 3.21 (1.20, 8.56) | 0.020 | |
| | Cytogenetic | 49/126 (39%) | 5.23 (1.68, 16.24) | 0.004 | |
| | Abnormalities | | | | |
| | Baseline 70-gene high | 27/127 (21%) | 5.18 (1.92, 13.94) | 0.001 | |
| | risk | | | | |
| | GEP TP53 deletion | 12/128 (9%) | 5.60 (1.94, 16.15) | 0.001 | |
| | Proliferation index high | 14/127 (11%) | 5.03 (1.80, 14.08) | 0.002 | |
| | Cl3 | 58/127 (46%) | 3.72 (1.20, 11.54) | 0.023 | |
| | Baseline 80-gene high | 15/128 (12%) | 6.74 (2.50, 18.16) | <.001 | |
| | risk | | | | |
| | Post bortezomib 80 | 22/128 (17%) | 8.82 (3.20, 24.34) | <.001 | |
| | gene high risk | | | | |
| Multivariate | GEP TP53 deletion | 11/122 (9%) | 3.49 (1.16, 10.51) | 0.027 | 26 |
| | Post bortezomib 80 | 22/122 (18%) | 6.69 (2.34, 19.12) | <.001 | 53 |
| | gene high risk | | | | |

### Testing in Other Protocol or Disease Settings

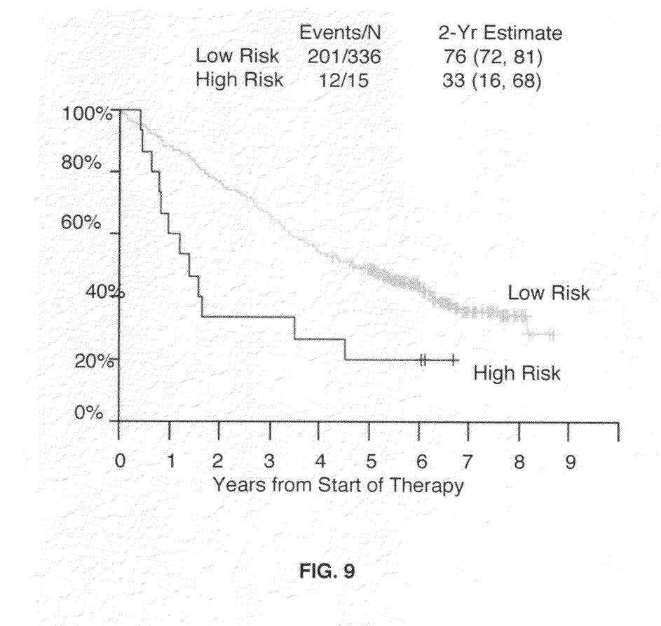

The notion that the baseline 80-gene score overcame the baseline 70-gene score in the test set suggests that it is a powerful baseline risk score for MM and can be used when the 48 hr expression is not available. The baseline 80-gene model on Total Therapy 2 (98-026; n=351) has been tested and the predicted high and low-risk groups exhibited significant difference in both event-free survival shown (FIG. 9) and OS (data not shown). Having already reported that the 70-gene score effectively distinguished post-relapse survival risk in TT2, the 80-gene model was also tested in that setting, and indeed the 80-gene score at relapse was as predictive of post-relapse survival as the 70-gene score (both p<0.001). However, when tested in diffuse large B-cell lymphoma and follicular lymphoma, neither the continuous nor binary 80-gene scores were associated with survival, suggesting that the 80-gene model may not be generalized to cancers other than MM.

### Example 2

### Proteasome Gene Up-Regulation is Unique to Bortezomib and was not Observed after Test-Dosing with Dexamethasone and Thalidomide in Total Therapy 2 (1998-26, TT2), and after Melphalan (2008-1, Total Therapy 4 [TT4])

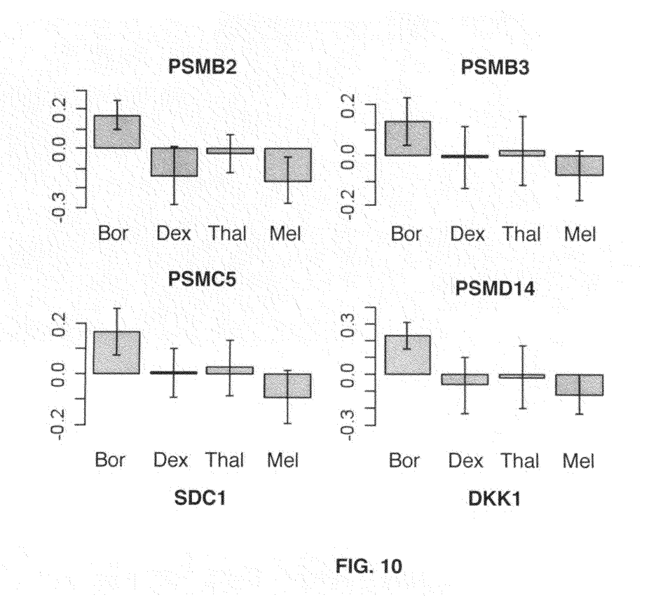

In earlier studies, pharmacogenomics was used to identify genes altered by short-term exposure of therapy-naïve cases to thalidomide and dexamethasone. Little to no overlap was found in the genes altered by these agents with those altered by bortezomib. The most obvious difference was the complete lack of activation of genes coding for proteasome components following thalidomide or dexamethasone exposure (FIG. 10). In the case of melphalan PG performed as part of Total Therapies 4, most proteasome genes did not change except PSMB2 and PSMD14 which were inversely changed compared to the changes after bortezomib. These data indicate that the bortezomib-induced gene alterations were unique to this first-in-class novel agent that were associated with poor prognosis linked to hyper-activation of its own target (proteasome) genes.

### DISCUSSION

The present invention validated that, within 48 hours of test-dose application, bortezomib induces hyper-activation of proteasome genes at the expense of gene subunits of the immuno-proteasome, which was associated with short event-free and overall survival in TT3, independent of 70-gene-derived risk designation. In fact, the 80-gene-derived risk model modified that provided by the 70-gene model in that low-risk patients were up-staged and high-risk patients down-staged. Further validated at the proteomics level, the proteasome hyper-activation involved cases in which a critical baseline expression level of proteasome genes was already present. This also led to the application of the 80-gene-derived post-bortezomib model to baseline and, importantly, the 80-gene-derived baseline model distinguished high-risk in 9% of subjects. However, when competing with the post-bortezomib-derived 80-gene model, the baseline high-risk 80-gene designation did not survive in the multivariate analysis (Table 3).

In comparative pharmacogenomic investigations of thalidomide and dexamethasone in TT2, and melphalan (10 mg/m2) test-dosing in Total Therapy 4, the bortezomib-induced gene alterations were shown to be drug-unique. The hyper-activation of proteasome genes after short-term exposure to bortezomib suggests a novel mechanism of resistance to this drug that was also discernable at baseline. In view of similar times of onset to and levels of CR among 80-gene-defined risk groups, the inferior outcome of such patients, as in the 70-gene model, reflects myeloma re-growth during treatment-free phases of the protocol, thus providing the basis for dose-dense and less dose-intense therapy currently under investigation in Total Therapy 5.

The following reference is cited herein:
1. Shaughnessy et al. Blood, 109, 2276-2284 (2007).

What is claimed:

1. A method for predicting a likelihood of transformation from a low-risk prognosis to a high-risk prognosis for a subject with multiple myeloma, comprising:
obtaining a gene expression profile of tumor cell genes before administration of a chemotherapeutic agent
administering a single dose of a chemotherapeutic agent to the subject;
obtaining a gene expression profile of tumor cell genes after administration of the chemotherapeutic agent; and
comparing the before and after gene expression profiles, wherein upregulation of genes in the profile obtained after administration compared to the profile before administration is indicative of a likelihood of transformation to a high-risk prognosis.

2. The method of claim 1, wherein the genes are selected from the group consisting of COX6C, NOLA1, COPS5, SOD1, TUBA6, HNRPC, PSMB2, PSMC4, LOC400657, C1orf31, FUNDC1, SUMO1, PSMB4, PSMB3, ENSA, PSMB4, COMMD8, MRPL47, PSMC5, PSMA4, PSMD4, NMT1, PSMB7, NXT2, SLC25A14, PSMD2, SNRPD1, CHORDC1, PSMD14, LAP3, PSMA7, UBPH, BIRC5, STAU2, ALDOA, TMC8, C1orf128, FLNA, HIST1H3B.

3. The method of claim 1, further comprising assigning a score based on the correlation of the upregulated genes expression profile to a risk of transformation in the prognosis for the subject.

4. The method of claim 3, wherein the risk of transformation is determined using multivariate analyses.

5. The method of claim 1, further comprising designing a therapeutic regimen effective to prevent transformation to the high-risk state by suppressing the hyperactivation of said upregulated genes.

6. The method of claim 1, wherein the tumor cell genes are proteosome genes.

7. The method of claim 1, wherein the chemotherapeutic agent is bortezomib.

8. The method of claim 1, wherein gene expression is determined at the nucleic acid level or at the protein level.

9. The method of claim 1, wherein the gene expression profile after administration of the chemotherapeutic agent is obtained in about 48 hours.

10. A method for identifying latently aggressive multiple myeloma tumor cells in a subject, comprising;
obtaining a first gene expression profile of multiple myeloma tumor cells in the subject;
contacting the tumor cells with a single chemotherapeutic agent; and
obtaining a second gene expression profile of the multiple myeloma tumor cells after contact with the chemotherapeutic agent; wherein upregulation of genes in the second profile compared to the first profile is indicative that the tumor cells are latently aggressive tumor cells.

11. The method of claim 10, wherein the genes are selected from the group consisting of COX6C, NOLA1, COPS5, SOD1, TUBA6, HNRPC, PSMB2, PSMC4, LOC400657, C1orf31, FUNDC1, SUMO1, PSMB4, PSMB3, ENSA, PSMB4, COMMD8, MRPL47, PSMC5, PSMA4, PSMD4, NMT1, PSMB7, NXT2, SLC25A14, PSMD2, SNRPD1, CHORDC1, PSMD14, LAP3, PSMA7, UBPH, BIRC5, STAU2, ALDOA, TMC8, C1orf128, FLNA, HIST1H3B.

12. The method of claim 10, further comprising predicting a likelihood that the subject will transform to a high-risk prognosis based on the level of gene activation in the second profile.

13. The method of claim 10, wherein the tumor cell genes are proteosome genes.

14. The method of claim 10, wherein the chemotherapeutic agent is bortezomib.

15. The method of claim 10, wherein gene expression is determined at the nucleic acid level or at the protein level.

16. The method of claim 10, wherein the second gene expression profile is obtained about 48 hours after administration of the chemotherapeutic agent.

17. A method for treating multiple myeloma in a subject, comprising:
determining if the multiple myeloma tumor cells in the subject are latently aggressive;
designing a chemotherapeutic regimen comprising one or more anticancer agents effective to suppress activation of genes in the latently aggressive tumor cells; and
administering the one or more anticancer agents to the subject thereby treating the multiple myeloma.

18. The method of claim 17, wherein determining if the multiple myeloma tumor cells are latently aggressive comprises:
obtaining a first gene expression profile of multiple myeloma tumor cells in the subject;
contacting the tumor cells with a single chemotherapeutic agent; and
obtaining a second gene expression profile of the multiple myeloma tumor cells after contact with the chemotherapeutic agent; wherein activation of genes in the second profile compared to the first profile is indicative that the tumor cells are latently aggressive.

19. The method of claim 18, wherein the genes are selected from the group consisting of COX6C, NOLA1, COPS5, SOD1, TUBA6, HNRPC, PSMB2, PSMC4, LOC400657, C1orf31, FUNDC1, SUMO1, PSMB4, PSMB3, ENSA, PSMB4, COMMD8, MRPL47, PSMC5, PSMA4, PSMD4, NMT1, PSMB7, NXT2, SLC25A14, PSMD2, SNRPD1, CHORDC1, PSMD14, LAP3, PSMA7, UBPH, BIRC5, STAU2, ALDOA, TMC8, C1orf128, FLNA, HIST1H3B.

20. The method of claim 18, wherein the tumor cell genes are proteosome genes.

21. The method of claim 18, wherein the chemotherapeutic agent is bortezomib.

22. The method of claim 18, wherein gene expression is determined at the nucleic acid level or at the protein level.

23. The method of claim 18, wherein the second gene expression profile is obtained about 48 hours after administration of the chemotherapeutic agent.

24. The method of claim 18, wherein the anticancer agent is bortezomid or thalidomide or combination thereof.

25. A method for predicting a likelihood of transformation from a low-risk prognosis to a high-risk prognosis for a subject with multiple myeloma, comprising:
administering a single dose of a chemotherapeutic agent to the subject;
obtaining a gene expression profile of tumor cell genes before and after administration of the chemotherapeutic agent; and
comparing the before and after gene expression profiles; wherein downregulation of genes in the profile obtained after administration compared to the profile before administration is indicative of a likelihood of transformation to a high-risk prognosis.

26. The method of claim 25, wherein the downregulated genes are selected from a group consisting of FOSB, LOC644250, C17orf60, LZTR2, PDE4B, STAU2, PDE4B, GABARAPL1, TAGAP, LOC643318, CISH, NR4A1, MGC61598, ANKRD37, KIAA1394, ACVR1C, TBC1D9, CRYGS, PDE4B, ZNF710, RBM33, STX11, KIAA1754, RPL41, WIRE, LAPTM4A, KLHL7, C9orf130, C14orf100.

27. The method of claim 25, further comprising assigning a score based on the correlation of the downregulated genes expression profile to a risk of transformation in the prognosis for the subject.

28. The method of claim 27, wherein the risk of transformation is determined using multivariate analyses.

29. The method of claim 25, further comprising designing a therapeutic regimen effective to prevent transformation to the high-risk state by hyperactivating of said down-regulated genes.

30. The method of claim 25, wherein the chemotherapeutic agent is bortezomib.

31. The method of claim 25, wherein gene expression is determined at the nucleic acid level or at the protein level.

32. The method of claim 25, wherein the gene expression profile after administration of the chemotherapeutic agent is obtained in about 48 hours.

33. A method for identifying latently aggressive multiple myeloma tumor cells in a subject, comprising;
obtaining a first gene expression profile of multiple myeloma tumor cells in the subject;
contacting the tumor cells with a single chemotherapeutic agent; and
obtaining a second gene expression profile of the multiple myeloma tumor cells after contact with the chemotherapeutic agent; wherein downregulation of genes in the second profile compared to the first profile is indicative that the tumor cells are latently aggressive tumor cells.

34. The method of claim 33, wherein the genes are selected from the group consisting of FOSB, LOC644250, C17orf60, LZTR2, PDE4B, STAU2, PDE4B, GABARAPL1, TAGAP, LOC643318, CISH, NR4A1, MGC61598, ANKRD37, KIAA1394, ACVR1C, TBC1D9, CRYGS, PDE4B, ZNF710, RBM33, STX11, KIAA1754, RPL41, WIRE, LAPTM4A, KLHL7, C9orf130, C14 orf 100.

35. The method of claim 33, further comprising predicting a likelihood that the subject will transform to a high-risk prognosis based on the level of gene suppression in the second profile.

36. The method of claim 33, wherein the chemotherapeutic agent is bortezomib.

37. The method of claim 33, wherein gene expression is determined at the nucleic acid level or at the protein level.

38. The method of claim 33, wherein the second gene expression profile is obtained about 48 hours after administration of the chemotherapeutic agent.

39. A method for treating multiple myeloma in a subject, comprising:
determining if the multiple myeloma tumor cells in the subject are latently aggressive;
designing a chemotherapeutic regimen comprising one or more anticancer agents effective to suppress activation of genes in the latently aggressive tumor cells; and
administering the one or more anticancer agents to the subject thereby treating the multiple myeloma.

40. The method of claim 39, wherein determining if the multiple myeloma tumor cells are latently aggressive comprises:
obtaining a first gene expression profile of multiple myeloma tumor cells in the subject;
contacting the tumor cells with a single chemotherapeutic agent; and
obtaining a second gene expression profile of the multiple myeloma tumor cells after contact with the chemotherapeutic agent; wherein suppression of genes in the second profile compared to the first profile is indicative that the tumor cells are latently aggressive.

41. The method of claim 39, wherein the genes are selected from the group consisting of FOSB, LOC644250, C17orf60, LZTR2, PDE4B, STAU2, PDE4B, GABARAPL1, TAGAP, LOC643318, CISH, NR4A1, MGC61598, ANKRD37, KIAA1394, ACVR1C, TBC1D9, CRYGS, PDE4B, ZNF710, RBM33, STX11, KIAA1754, RPL41, WIRE, LAPTM4A, KLHL7, C9orf130, C14orf100.

42. The method of claim 40, wherein the chemotherapeutic agent is bortezomib.

43. The method of claim 40, wherein gene expression is determined at the nucleic acid level or at the protein level.

44. The method of claim 40, wherein the second gene expression profile is obtained about 48 hours after administration of the chemotherapeutic agent.

45. The method of claim 40, wherein the anticancer agent is bortezomid or thalidomide or combination thereof.
